(19) United States
(12) Patent Application Publication
(10) Pub. No.: US 2023/0071731 A1
(43) Pub. Date: Mar. 9, 2023

(54) SYSTEMS AND METHODS FOR CONTROLLING AND MONITORING ENVIRONMENTAL CONDITIONS OF A SEMEN SAMPLE DURING TRANSPORT

(71) Applicant: Fellow Health Inc., San Francisco, CA (US)

(72) Inventors: Owen Falk, San Francisco, CA (US); William Matthews, San Francisco, CA (US)

(21) Appl. No.: 17/797,091

(22) PCT Filed: May 27, 2021
(86) PCT No.: PCT/US2021/034628

§ 371 Date: Aug. 2, 2022

Related U.S. Application Data
(60) Provisional application No. 63/030,730, filed on May 27, 2020.

(51) Int. Cl. A01N 1/02

(52) U.S. Cl. CPC A01N 1/0252; A01N 1/0284; A01N 1/021

(57) ABSTRACT

Provided herein are systems, methods, and kits for controlling and monitoring environmental conditions of a semen sample during transport. The technologies disclosed herein allow for at-home male fertility sample collection and subsequent transport to a laboratory to undergo fertility testing. One or more environmental conditions can be tracked during transport which provide the data used to determine whether a sample needs to be resubmitted or if the conditions during transport were such that a false test result is unlikely.

### CROSS-REFERENCE

The present application is a U.S. National Phase application, under 35 U.S.C. § 371, claiming priority to PCT Application No. PCT/US2021/034628, filed May 27, 2021, entitled “Systems and Methods for Controlling and Monitoring Environmental Conditions of a Semen Sample During Transport,” which claims priority to U.S. Provisional Patent Application No. 63/030,730, filed May 27, 2020, entitled, “Semen Analysis Kit”, the disclosures of both are incorporated by reference herein in their entireties for all purposes.

### FIELD OF THE INVENTION

This description is generally directed toward systems, methods, and kits for controlling and monitoring environmental conditions of male fertility samples during transport and determining whether the male fertility samples are suitable for undergoing male fertility testing.

### BACKGROUND

Currently, male fertility testing includes semen sample collection at a fertility clinic or doctor's office. Samples are typically tested within one hour of collection at the same facility they were collected at.

Maintaining semen sample collection facilities at fertility clinics and doctor's offices can be expensive and uncomfortable. Many fertility clinics and doctor's offices are unable to dedicate space to semen sample collection, so patients are required to produce samples in undesirable places such as public restrooms or nearby hotels to still enable the sample to be tested within the one-hour processing window.

Semen sample collection requires comfort and relaxation, whereas, clinics and doctor's offices often foster discomfort and anxiety in people. As such, there is a need for systems, methods, and kits allowing for sample collection for fertility testing to occur in one's home while providing individualized transport in an environmentally controlled and monitored manner. The present disclosure addresses this and other needs.

### SUMMARY

In some aspects, an environmental control and monitoring system for transportation and analysis of a semen sample is disclosed. In some embodiments, the system may comprise a sample container, configured to contain a semen sample, an environmental control element configured to operate within an optimal range for semen viability, and a sensor system for monitoring an environmental condition of the sample container, wherein the sensor system is in thermal contact with at least one of the sample container or the environmental control element.

In some embodiments, the system further comprises a positioning retainer for restraining movement of the sample container, the environmental control element, and the sensor relative to one another. In some embodiments, the positioning retainer comprises a packing material.

In some embodiments, the sensor system may further comprise a sensor for measuring the environmental condition, a data store in electronic communication with the sensor, wherein the data store is configured to store measurements from the sensor, and a data port in electronic communication with the data store. In some embodiments, the sensor comprises a temperature sensor and the environmental condition comprises temperature. In some embodiments, the sensor comprises a humidity sensor and the environmental condition comprises humidity. In some embodiments, the sensor measures the environmental condition at least one time every minute.

In some embodiments, the measurements from the sensor system may be stored on the data store. In some embodiments, the data port may electronically communicate measurements from the sensor system to a remote server. In some embodiments, the measurements may be stored on the data store prior to being transmitted to the remote server.

In some embodiments, the remote server may comprise a server data port for sending and receiving data, wherein the data comprises the measurements, a server data store in electronic communication with the server data port for storing the data, and a sever processor for analyzing the data.

In some embodiments, the environmental control element comprises a phase transition material. In some embodiments, the phase transition material may be configured to go through a phase change within the optimal temperature range. In some embodiments, the optimal range comprises temperatures ranging from 4° C. to 37° C.

In some embodiments, the environmental control and monitoring system may further comprise a container wall surrounding an internal cavity, wherein a positioning retainer fits within the internal cavity, wherein the cavity is surrounded by the container wall.

In some embodiments, the sample container may hold a semen preservation solution.

In some embodiments, the data port and the server data port each comprise a wireless adaptor.

In some embodiments, the sensor system comprises a temperature indicator. In some embodiments, the indicator may display a level of exposure. In some embodiments, the temperature indicator may be in physical and thermal contact with an exterior surface. In some embodiments, the temperature indicator may be in physical and thermal contact with an interior surface.

In some embodiments, the temperature indicator may be in physical and thermal contact with a retainer surface.

In some aspects, an environmental control and monitoring method for transportation and analysis of a semen sample is disclosed. In some embodiments, the method may comprise the steps of transporting a sensor system, an environmental control element, and a sample container, wherein the sensor system may be in thermal contact with at least one of the sample container or the environmental control element. In some embodiments, the method may comprise the step of controlling one or more environmental conditions of the sample container during transport using the environmental control element. In some embodiments, the method may comprise the step of recording a plurality of measurements of the one or more environmental conditions during transport of the sample container to a log. In some embodiments, the method may comprise the step of comparing the log to a reference to determine a condition of the sample container.

In some embodiments, the method may further comprise the step of introducing a semen sample into the sample container prior to the transporting step.

In some embodiments, the method may further the step of registering the semen sample with a laboratory to associate a subject with the semen sample. In some embodiments, the one or more environmental conditions comprises temperature. In some embodiments, the one or more environmental conditions comprises humidity. In some embodiments, the plurality of measurements may be recorded every 1, 2, 3, 4, 5, 6, 7, 8, 9, or 10 minutes. In some embodiments, the log comprises a plurality of temperature and timestamp pairs. In some embodiments, the reference comprises off-target temperature values. In some embodiments, the reference comprises exposure durations for the off-target temperature values. In some embodiments, the reference is based on published research data. In some embodiments, the reference is based on data acquired using the environmental control and monitoring method.

In some embodiments, the condition determines whether the sample undergoes fertility testing and is based on sperm count, motility count, semen volume, or morphology.

In some embodiments, the method further comprises causing a phase transition material of the environmental control element to undergo a phase transition. In some embodiments, the phase transition may occur between 18-24° C. In some embodiments, the environmental control element comprises a flexible material containing the phase transition material. In some embodiments, the flexible material comprises a 0.5 mm plastic film. In some embodiments, the phase transition material comprises a salt solution. In some embodiments, the phase transition material comprises a solution comprising sugar, NaCl, oil, glucose, water, mineral spirits, or a surfactant. In some embodiments, the environmental control element comprises a mass between 90-110 grams.

In some embodiments, the log comprises a temperature indicator. In some embodiments, the temperature indicator comprises an adhesive strip including a temperature sensitive region, wherein the temperature sensitive region gradually changes appearance based on duration of exposure to off-target temperature values. In some embodiments, the environmental control and monitoring method further comprising the steps of storing the log on a data store in digital format, transmitting the log from the data store to a remote server, and completing the comparing step on the remote server.

In some embodiments, the step of transporting comprises traveling more than 10 miles. In some embodiments, the step of transporting comprises traveling more than 10 minutes.

In some aspects, a kit for transporting a semen sample is disclosed. In some embodiments, the kit may comprise a sample container for storing a semen sample, an environmental control element for controlling a temperature of the sample container, and a sensor system for monitoring the temperature of the sample container.

In some embodiments, the sensor system of the kit may comprise a temperature sensor, a data store for recording measurements produced by the temperature sensor, and a data port for transmitting the measurements.

In some embodiments, the kit further comprises a positioning retainer for restraining movement of the sample container, the environmental control element, and the sensor system relative to one another.

In some embodiments, the kit further comprises a preservation container comprising a preservation fluid.

In some embodiments, the sensor system comprises an appearance changing temperature indicator.

In some embodiments, the appearance change involves a material going through a chemical transition after exposure to an off-target temperature or pH.

In some embodiments, the temperature control element comprises a phase transition material. In some embodiments, the phase transition material has been optimized to operate within a validated range for semen sample viability.

In some embodiments, the environmental control element may be a gel pack. In some embodiments, the phase change occurs between 18-24° C. In some embodiments, the environmental control element comprises a flexible material containing the phase transition material. In some embodiments, the flexible material comprises a 0.5 mm plastic film. In some embodiments, the phase transition material comprises a salt solution. In some embodiments, the phase transition material comprises a solution comprising sugar, NaCl, oil, glucose, water, mineral spirits, or a surfactant. In some embodiments, the environmental control element comprises a mass between 90-110 grams.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF FIGURES

The novel features of the technology are set forth with particularity in the appended claims. A better understanding of the features and advantages of the present technology will be obtained by reference to the following detailed description that sets forth illustrative embodiments, in which the principles of the technology are utilized, and the accompanying drawings (also “Figure” and “FIG.” herein). The accompanying drawings are not intended to be drawn to scale. Like reference numbers and designations in the various drawings indicate like elements. For purposes of clarity, not every component can be labeled in every drawing. In the drawings:

FIG. 1 is a top down view of an environmental control and monitoring system according to some embodiments.

FIG. 2 is a side view (wall removed for visibility) of an environmental control and monitoring system with a sensor system in thermal contact with an environmental control element according to some embodiments.

FIG. 3 shows a schematic diagram of a data and analysis system according to some embodiments.

FIG. 4 shows a schematic diagram of a sensor system according to some embodiments.

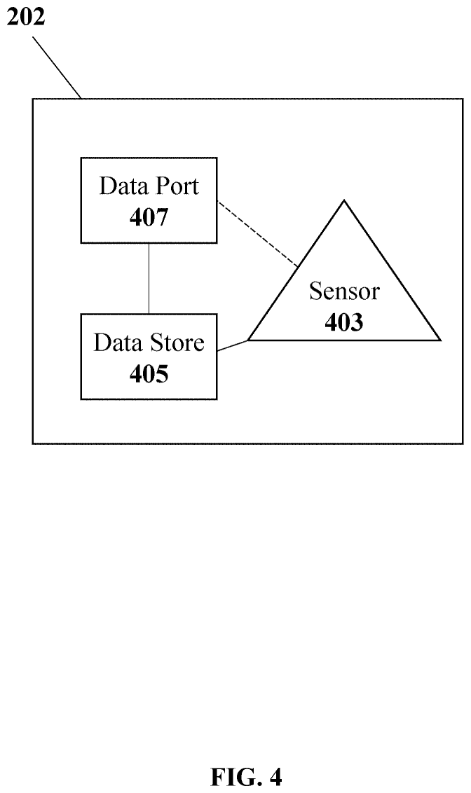

FIG. 5 shows a schematic diagram of a computer system according to some embodiments.

FIG. 6 is a side view (wall removed for visibility) of an environmental control and monitoring system with a sensor system on an exterior surface according to some embodiments.

FIG. 7 is a side view (wall removed for visibility) of an environmental control and monitoring system with a sensor system in thermal contact with a sample container according to some embodiments.

FIG. 8 is a side view (wall removed for visibility) of an environmental control and monitoring system with a sensor system in thermal contact with a retainer surface according to some embodiments.

FIG. 9 is a temperature indicator according to some embodiments.

FIG. 10 is a temperature indicator according to some embodiments.

FIG. 11 is an illustration of a flow chart providing an overview of the technologies described herein according to some embodiments.

FIG. 12 is a flow chart illustrating a method of controlling and monitoring environmental conditions of a thermal mass (e.g. contents within a package) according to some embodiments.

FIG. 13 is a flow chart illustrating a method of controlling and monitoring environmental conditions, using a smart sensor system, of a thermal mass (e.g. contents within a package) according to some embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

This specification describes various exemplary embodiments of systems, methods and kits for male fertility sample collection, transport, and analysis. The disclosure, however, is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion.

In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

It should be understood that any uses of subheadings herein are for organizational purposes and should not be read to limit the application of those subheaded features to the various embodiments herein. Each and every feature described herein is applicable and usable in all the various embodiments discussed herein and that all features described herein can be used in any contemplated combination, regardless of the specific example embodiments that are described herein. It should further be noted that exemplary descriptions of specific features are used, largely for informational purposes, and not in any way to limit the design, subfeature, and functionality of the specifically described feature.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, molecular biology, pharmacology and toxicology are described herein are those available and commonly used in the art.

### Definitions

As used herein, the terms “comprise”, “comprises”, “comprising”, “contain”, “contains”, “containing”, “have”, “having” “include”, “includes”, and “including” and their variants are not intended to be limiting, are inclusive or open-ended and do not exclude additional, unrecited additives, components, integers, elements or method steps. For example, a process, method, system, composition, kit, or apparatus that comprises a list of features is not necessarily limited only to those features but may include other features not expressly listed or inherent to such process, method, system, composition, kit, or apparatus.

As used herein, the term “environmental condition” can refer to any measurable condition within an environment. For example, temperature, pressure, and humidity are non-limiting examples of environmental conditions. Environmental conditions may comprise an abundance of something such as a contaminant, radioactive element, atom, or molecule. Environmental mental conditions can be measured over time. For example, an environmental condition (e.g. temperature) may be measured at a specified time frequency (e.g. every one minute).

As used herein, the term “packing material” can refer to any kind of material suitable for restricting movement of one or more objects contained within a box, package, other container during transport. Examples of packing materials may include cardboard, plastic, or other rigid materials that can be shaped or folded to a specification. For example, packing material may comprise one or more cardboard inserts designed to fit into packages or boxes. In some embodiments, in addition or alternate to the rigid materials, examples of packing materials may include paper, bubble wrap, or packing tape. For example, two or more objects may be held in close proximity by packing, sealing, attaching (e.g. using an adhesive material such as adhesive coated plastic film). In some embodiments, packing materials may restrict movement of objects (e.g. a sample container) within a package by applying physical or elastic force, attachment via adhesive, compression forces (e.g. paper packing tightly with objects within a container).

As used herein, the term “phase transition material” or “phase change material” means any material that relies on a phase change (e.g. solid to liquid) to release or absorb energy to maintain a temperature range. In some embodiments, a phase change material can be adjusted to work within a specified range. In some embodiments, phase change materials can be optimized to maintain viability of a semen sample. In some embodiments, plastic containers, plastic bags, or gel packs may be filled with phase transition material and used to control temperatures in confined spaces such as packages (e.g. packages containing a thermal mass or kit components described herein).

As used herein, the term “plurality” can be 2, 3, 4, 5, 6, 7, 8, 9, 10, or more.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

As used herein, the term “thermal contact” means two or more systems or objects capable of exchanging energy through the process of heat. In the kits described below the temperature system, environmental control element, and sample container are often described as being in thermal contact. Generally, objects that are in thermal contact with one another converge to the same or a similar temperature over time. Objects may be in thermal contact with one another through physical contact with an intermediary (such as another object) or be in direct physical contact.

As used herein, the term “thermal mass” means one or more systems or objects in thermal contact with one another. For example, in some embodiments herein, a thermal mass may comprise a sample container, an environmental control element, and a sensor system. In some embodiments, one or more walls and/or surfaces may be part of the thermal mass. In some embodiments, a positioning retainer may be part of the thermal mass.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular.

### I. Overview

Environmental control and monitoring systems can aid in male fertility sample transport after collection and can collect data that determines whether a fertility sample is a suitable candidate for fertility testing. In some embodiments, a patient may deposit a semen sample into a collection container for transport. A known issue within the industry is that collection of semen samples under stressful conditions, such as how subjects feel in hospital or clinic settings can negatively impact sperm (e.g. reduce motility, volume, negatively impact morphology). An advantage of the embodiments of the present disclosure is patients being able to complete the deposit of a collection sample in the comfort of their own home and then being able to prepare the sample for transport. A major hurdle facing the industry is having an effective way to environmentally control and monitor the environment (e.g. an environmental condition) within a sample container and then be able to effectively communicate a history of the environmental conditions for laboratory analysis.

### II. Systems

Provided herein are systems for collection, transport, and analysis of male fertility samples. More specifically, the systems described herein allow for control and monitoring of the environmental conditions in a semen sample during transport. For example, it is important for laboratories to know the condition of samples before they arrive so that they can determine whether the sample is capable of producing viable results. In other words, sperm need to arrive undamaged due to off-target environmental conditions in order for accurate testing to occur. If a semen sample is subjected to temperatures outside of a preferred range for a long enough time period, then the sample is no longer viable and further testing will not reveal information relating to fertility. Alternatively, knowing that a sample was handled in a manner that will not disrupt (or predictably modify) motility, morphology of sperm or other aspects of semen health then further fertility testing can be completed without concern for environmental contributors impacting the results of the test.

In some aspects, systems, methods, and kits are provided allowing for environmentally controlled and monitored transport of male fertility samples. In some embodiments, systems, methods, and kits are provided allowing for initial conditions of fertility samples to be determined based predictable motility and morphology changes over time. As such, embodiments herein allow for male fertility samples to undergo varying time delays until testing while still being able to provide accurate results.

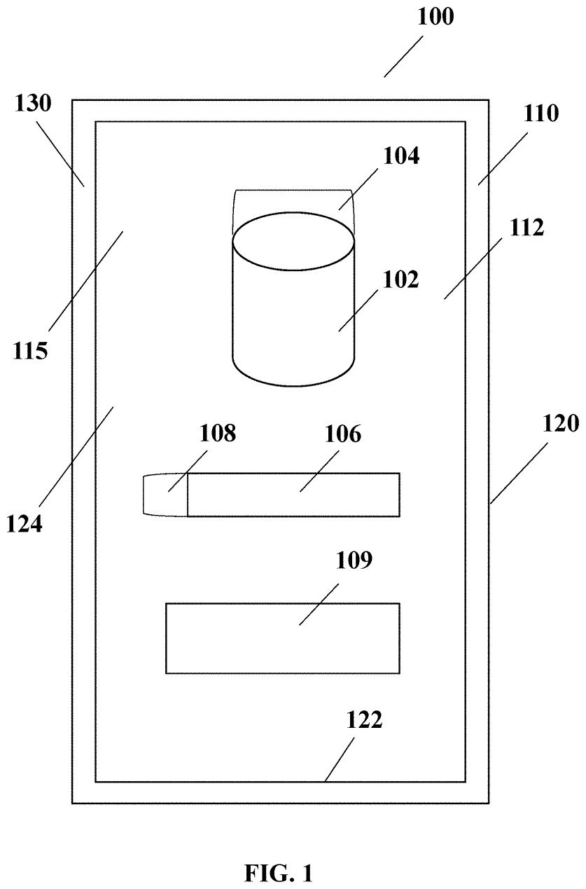

FIG. 1 is a top down view of an environmental control and monitoring system 100 according to some embodiments. In some embodiments, the environmental control and monitoring system 100 may comprise a container 130 having a container wall 110 surrounding an internal cavity 112. In some embodiments, the container wall 110 may have an exterior surface 120 and an interior surface 122.

In some embodiments, the container 130 can comprise a packing material such as cardboard or plastic. In some embodiments, the container 130 may be designed for shipping such that the walls may include internal insulation for reducing temperature fluctuations. In some embodiments, the packing material may be reinforced to prevent damage to the internal contents of the container 130. In some embodiments, insulation or reinforcing materials may be contained within the container wall 110 between the exterior surface 120 and the interior surface 122.

In some embodiments, an environmental control and monitoring system 100 may include a positioning retainer 115 that may fit into the container 130. In some embodiments, the positioning retainer 115 may be hold in place using friction. In some embodiments, the friction may occur between the interior surface 122 and some part of the positioning retainer 115. For example, in some embodiments, a retainer surface 124 may press against an interior surface 122 of the container 130, thereby, causing the positioning retainer 124 to be held in place using friction.

In some embodiments, the positioning retainer 124 may include a plurality of openings or cut-outs matching the profile of a sample container 102 and lid 104, a preservation container 106 and lid 108, and a biohazard bag 109. In some embodiments, cut-outs can be created to accommodate any physical object requiring shipment or temperature control and monitoring. In some embodiments, the positioning retainer 115 may be responsible for ensuring that the components in the container 130 do not jostle or move during transport.

In some embodiments, a container 130 may comprise a cardboard box. In some embodiments, a container 130 may comprise a paper or plastic bag or envelope. In some embodiments, the container 130 may comprise a hollow tube. In some embodiments, the container 130 may comprise a bin or barrel. In some embodiments, the container 130 may comprise an insulation material such as a hollow, vacuum sealed, wall.

In some embodiments, semen sample collection may occur in the comport of the patient's home. In some embodiments, the sample may be produced and contained within a sample container 102 for shipment. In some embodiments, collection follows a protocol minimizing contamination of the sample. In some embodiments, once the sample is deposited into the sample container 102, a sample lid 102 can be attached, affixed, or screwed onto the sample container 102. In some embodiments, a seal between the sample lid 104 and sample container 102 ensures the sample does not become contaminated during transport.

In some embodiments, the sample container 102 and sample lid 104 may comprise an insulated material to help regulate changes in temperature of the sample. In some embodiments, the sample container 102 may comprise a double wall with a vacuum in between the double wall to reduce heat transfer.

In some embodiments, the environmental control and monitoring system 100 may comprise a preservation container 106 including a preservation lid 108. In some embodiments, the preservation container 106 may ship to a patient and include a preservation solution. In some embodiments, the preservation solution may comprise salts, nutrients, or antibiotics. In some embodiments, the preservation solution may be added to the sample container 102 prior to sample collection. In some embodiments, the preservation solution may be added to the sample container 102 after sample collection. In some embodiments, the preservation solution may increase the likelihood of maintaining semen or sperm viability in a predictable manner during transport. In such embodiments, linear regression models or other predictive algorithms may be used to accurate predict a condition of a semen sample at the time of collection (See Examples Section).

In some embodiments, the environmental control and monitoring system 100 may comprise a biohazard bag 109. In some embodiments, the biohazard bag 109 may be used to contain the sample container 102 including the collected sample during transport. In some embodiments, the biohazard bag 109 may be an extra measure to ensure the fertility, semen, or sperm sample stays contaminant free during transport. In some embodiments, the biohazard bag 109 may comprise a flexible plastic material. In some embodiments, the biohazard bag 109 may comprise any kind of container capable of maintaining a sterile environment within its interior.

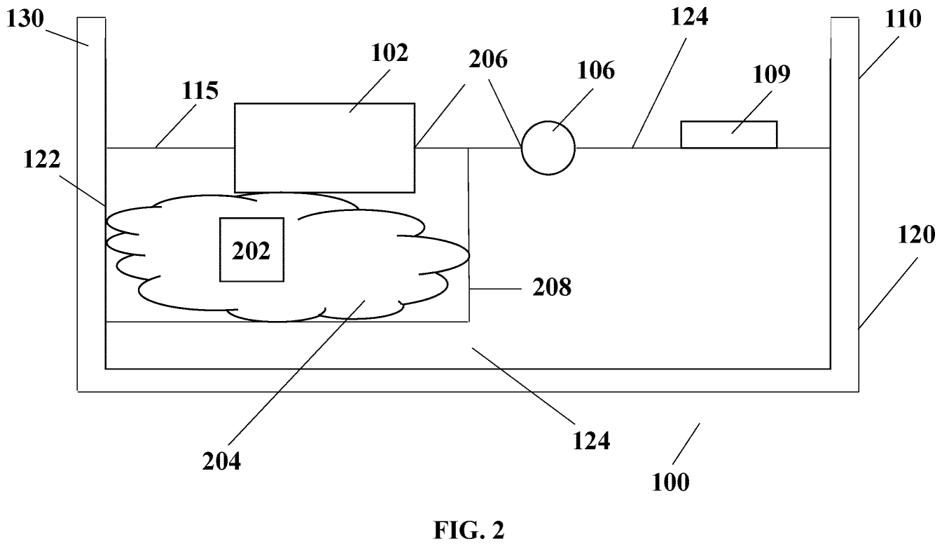

FIG. 2 is a side view (wall removed for visibility) of an environmental control and monitoring system 100 with a sensor system 202 in thermal contact with an environmental control element 204 according to some embodiments. Referring to FIG. 2, a positioning retainer 115 is shown fitting inside of a container 130 within a container wall 110. In some embodiments, a positioning retainer 115 may have cut-outs 206 in the retainer surface 115 so that objects can be fitted into the positioning retainer 115 such that their movement is restrained during transport or movement of any kind. In some embodiments, objects may rest on top of the retainer surface 124. In some embodiments, the positioning retainer may include additional flaps, hooks, or securing components to prevent objects from moving during transport. In some embodiments, the objects being restrained may include a sample container 102, a preservation container 106, a biohazard bag 109, a sensor system 202, and an environmental control element 204.

In some embodiments, the environmental control and monitoring system 100 comprises an environmental control element 204, a sensor system 202, and a sample container 102 all being part of the same thermal mass. For example, in some embodiments, heat transfers easily between the environmental control element 204, the sensor system 202, and the sample container 102. In such embodiments, the environmental control element 204, the sensor system 202, and the sample container 102 are in thermal contact.

For example, in some embodiments, a sensor system 202 may be in direct physical contact with an environmental control element 204 and in thermal contact with both the environmental control element 204 and a sample container 102. In some embodiments, a sensor system 202 may be in direct physical contact with a sample container 102 and in thermal contact with both the sample container 102 and the environmental control element 204. In some embodiments, an environmental control element 204, a sensor system 202, and a sample container 102 may all be in both physical and thermal contact with one another.

In some embodiments, a positioning retainer 115 may comprise an insert capable of fitting into a container wall 110 of a container 130. In some embodiments, a positioning retainer 115 may comprise a retainer surface 124. In some embodiments, a positioning retainer 115 comprise cut-outs 106, flaps, adhesive, hooks, or anything that may assist in retaining physical objects on, at, or underneath a retainer surface 124. In some embodiments, a positioning retainer may comprise positioning retainer material 208 that may be part of the retainer surface 124 or comprise a cut-out 206 or a flap.

In some embodiments, the positioning retainer 115 may be shaped to ensure a sample container 102, an environmental control element 204, and a sensor system 202 all remain in thermal contact. In some embodiments, the sensor system 202 takes temperature measurements every 1, 2, 3, 4, 5, 6, 7, 8, 9, or 10 minutes or at any other frequency. In some embodiments, the sensor system 202 takes continuous temperature measurements. In some embodiments, the sensor system 202 may indicate whether the sample has been exposed to out of range temperatures and a duration. For example, an indicator may report that a temperature was recorded at or above forty-two degrees for a certain amount of time.

Referring to FIG. 2, a depiction of a sample container 102 inserted into a cut-out 206 of a retainer surface 124 is shown. In some embodiments, a positioning retainer 115 holds the sample container 102 in physical contact with the environmental control element 204 so that thermal transfer occurs, keeping the sample within viable temperature range. In some embodiments, the positioning retainer 115 holds the environmental control element 204 in physical contact with the sensor system 202.

In some embodiments, environmental control elements 204 may comprise a phase transition material. In some embodiments, the phase transition material may be configured to operate within an optimal range for maintaining sperm viability.

In some embodiments, an optimal temperature range for a sample may be between 4° C.-37° C., 5° C.-36° C., 6° C.-35° C., 7° C.-34° C., 8° C.-33° C., 9° C.-32° C., 10° C.-31° C., 11° C.-30° C., 12° C.-29° C., 13° C.-28° C., 14° C.-27° C., 15° C.-26° C., 16° C.-25° C., 17° C.-24° C., 18° C.-23° C., 19° C.-22° C., or 20° C.-21° C.

Any of the configurations depicted in FIGS. 1, 2, 6, 7, and 8 may comprise smart sensor systems, indicator sensor systems, or both.

In some embodiments, environmental control elements may comprise a phase transition material. In some embodiments, the phase transition material may be formulated to operate within an optimal temperature range. In some embodiments, the phase transition material may comprise a container such as packaging. In some embodiments, the container may comprise a plastic film creating a compartment for container the phase transition material.

In some embodiments, the phase transition material may be formulated to complete a phase transition or phase change between about 15° C.-27, ° C., 16° C.-26° C., 17° C.-25° C., 18° C.-24° C., 19° C.-23° C., or 20° C.-22° C. or any combination thereof.

In some embodiments, the phase transition material may comprise a salt solution. In some embodiments, the phase transition material may comprise NaCl. In some embodiments, the phase transition material may comprise a sugar solution. In some embodiments, the phase transition material may comprise glucose. In some embodiments, the phase transition material may comprise water. In some embodiments, the phase transition material may comprise oil. In some embodiments, the phase transition material may comprise mineral spirits. In some embodiments, the phase transition material may comprise a surfactant.

In some embodiments, the environmental control element may comprise a mass of 100 grants. In some embodiments, the environmental control element may comprise a mass between about 95-105, 90-110, 85-115, grams or any combination thereof.

### Smart Sensor System

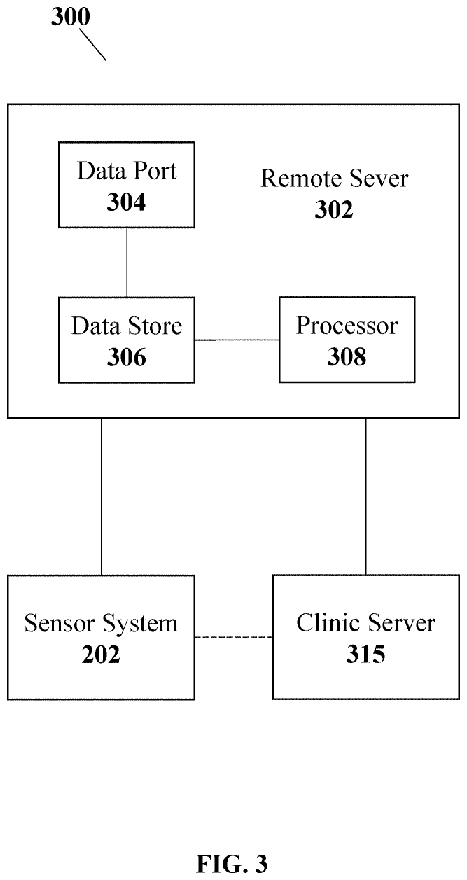

In some aspects of the disclosure, a smart sensor system is detailed. FIG. 3 shows a schematic diagram of a smart sensor system 300 according to some embodiments. In some embodiments, a smart sensor system 300 comprises a sensor system 202 and a remote sever 302. In some embodiments, a smart sensor system 300 comprises a sensor system 202, a remote sever 302, and a clinic sever 315.

In some embodiments, a sensor system 202 shares the same environmental conditions as a sample being transported and delivered to a laboratory for testing. In some embodiments, the sensor system 202 measures an environmental condition at a given time interval. For example, in some embodiments, a temperature reading may be taken ever one minute by the sensor system 202 and transmitted to a remote sever 302.

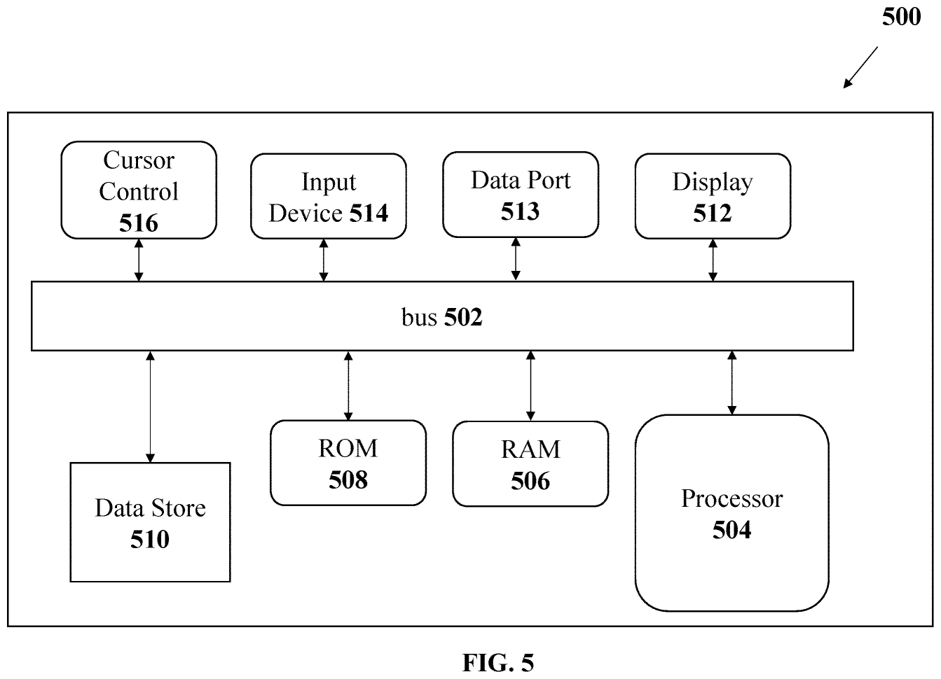

Aspects of the disclosure include embodiments for remote servers. In some embodiments, remote servers may comprise any device capable of computation. In some embodiments, a remote sever may comprise some or all of typical computer components such as those depicted in the computer system 500 in FIG. 5.

In some embodiments, a remote sever 302 may comprise a server data port 304 for sending and receiving information (e.g. environmental condition measurements), a server data store 306 for storing information, and a server processor 308 for processing data. In some embodiments, for example, the server processor 308 may receive data relating to an environmental condition (e.g. temperature) out of a setpoint for a duration and by an amount. The processor can use the data to determine whether the fertility sample is still viable for testing purposes.

In some embodiments, a clinic server 315 may comprise a data port for sending and receiving information, a data store for storing information, and a processor for processing data. In some embodiments, the clinic server 315 is a computer system in a doctor's office or a clinic configured to receive test results from the remote server 302.

In some embodiments, transmission of information between a sensor system 202, a remote server 302, and a clinic server 315 occur by known methods. For example, in some embodiments, the remove server 306, sensor system 202, and clinic server 315, each comprise a data port 304, 407. In some embodiments, the data ports 304, 407 comprise wireless internet adaptors (“WIFI”). In some embodiments, the data ports 304, 407 comprise ethernet adaptors. In some embodiments, the data ports 304, 407 comprise USB ports. In some embodiments, the data ports 304, 407 comprise a long-term storage medium such as a hard disk, floppy disk, compact disk, etc. In some embodiments, the data ports 304, 407 comprise anything that can transmit electronic data between two computer systems.

In some embodiments, an optimal temperature range for a sample may be between 4° C.-37° C., 5° C.-36° C., 6° C.-35° C., 7° C.-34° C., 8° C.-33° C., 9° C.-32° C., 10° C.-31° C., 11° C.-30° C., 12° C.-29° C., 13° C.-28° C., 14° C.-27° C., 15° C.-26° C., 16° C.-25° C., 17° C.-24° C., 18° C.-23° C., 19° C.-22° C., or 20° C.-21° C.

In some embodiments, environmental conditions can refer to any measurable condition within an environment. In some embodiments, an environmental condition may comprise temperature. In some embodiments, an environmental condition may comprise humidity. In some embodiments, an environmental condition may comprise pressure. In some embodiments, an environmental condition may comprise an abundance of a contaminant.

In some embodiments, data stores and server data stores may comprise any device capable of storing information in a non-transient manner. For example, in some embodiments, data stores may comprise hard disks, CD or optical read devices, or any other device capable of non-transient storage.

In some embodiments, data stores may comprise random access memory or other transient forms of storage.

In some embodiments, a sensor system 202 may comprise a data port 407, a data store 405, and a sensor 403 or sensing element.

In some embodiments, the sensor may comprise the device or apparatus conducting measurements. For example, in some embodiments, humidity, temperature, and pressure sensors may be purchase from known commercial manufactures.

In some embodiments, a data port 407 may comprise a WIFI adaptor, an ethernet port, a USB port, or any other wireless or hardwire port known to transmit electronic data.

In some embodiments, a data store 405 may comprise one or more hard disks, one or more CD or optical read devices, or any other device capable of non-transient storage.

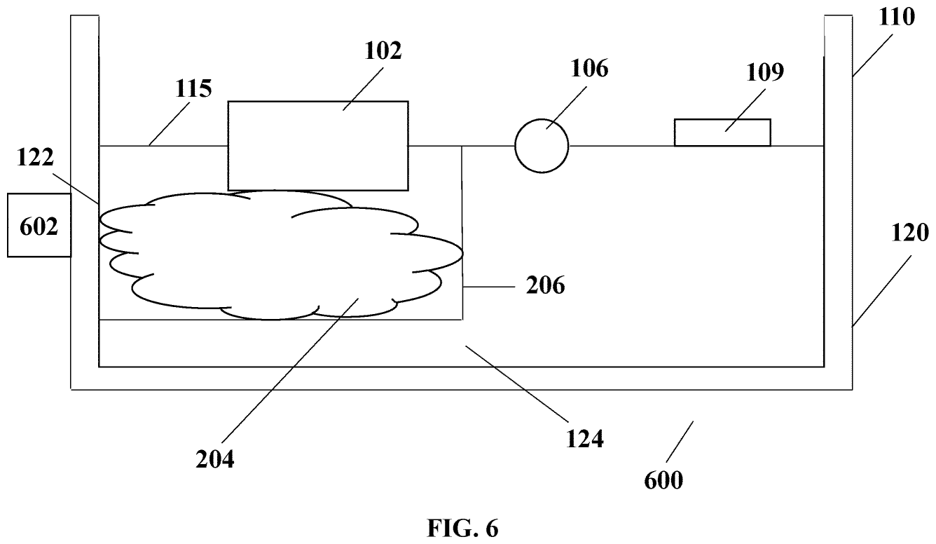

FIG. 6 is a side view (wall removed for visibility) of an environmental control and monitoring system 600 with a sensor system 602 in thermal contact with an environmental control element 204 according to some embodiments. In some embodiments, a sensor system 602 may be in thermal contact with an environmental control element 204 through an intermediary such as a container wall 110. In some embodiments, an environmental control element 204 may contact an interior surface 122 of a container wall 110 and a sensor system 602 may contact an exterior wall 120 of the container wall 110 on opposite sides of the container wall 110 such that a thermal mass comprises a portion of the container wall 110 in addition to the sensor system 602, environmental control element 204 and sample container 102. In some embodiments, the portion of the container wall 110 contacting the sensor system 602 and the environmental control element 204 may comprise a material having a high heat transfer efficiency.

In some embodiments, the sensor system 602 may span some or all of the container wall 110. In some embodiments, the sensor system 602 may be mounted to the exterior wall. In some embodiments, the sensor system 602 may be attached to the container wall 110 with adhesive, brackets, or any other known way to attach two objects.

Referring to FIG. 6, a positioning retainer 115 is shown fitting inside of a container 130 within a container wall 110. In some embodiments, a positioning retainer 115 may have cut-outs 206 in the retainer surface 115 so that objects can be fitted into the positioning retainer 115 such that their movement is restrained during transport or movement of any kind. In some embodiments, objects may rest on top of the retainer surface 124. In some embodiments, the positioning retainer may include additional flaps, hooks, or securing components to prevent objects from moving during transport. In some embodiments, the objects being restrained may include a sample container 102, a preservation container 106, a biohazard bag 109, a sensor system 602, and an environmental control element 204.

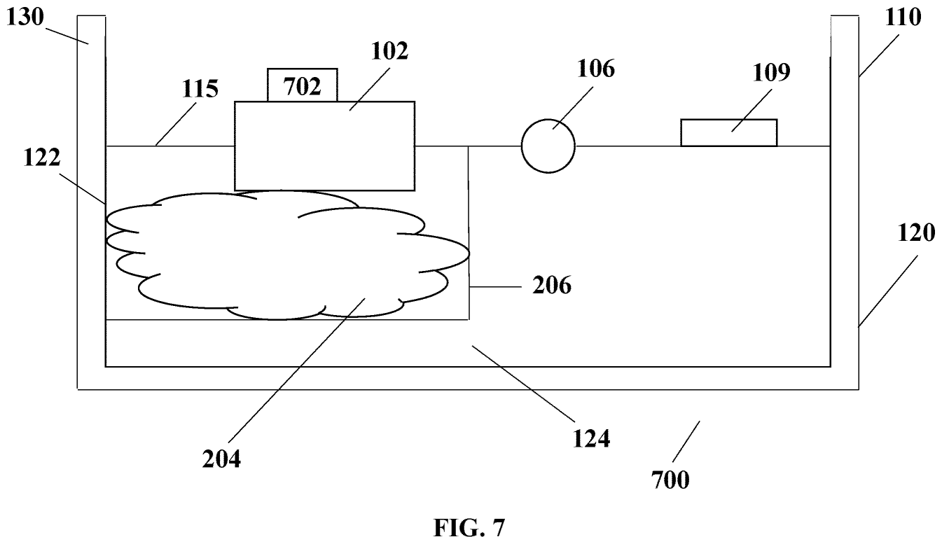

FIG. 7 is a side view (wall removed for visibility) of an environmental control and monitoring system 700 with a sensor system 702 in physical and thermal contact with a sample container 102 according to some embodiments. In some embodiments, a sensor system 702 may be in thermal contact with an environmental control element 204 through an intermediary such as the sample container 102.

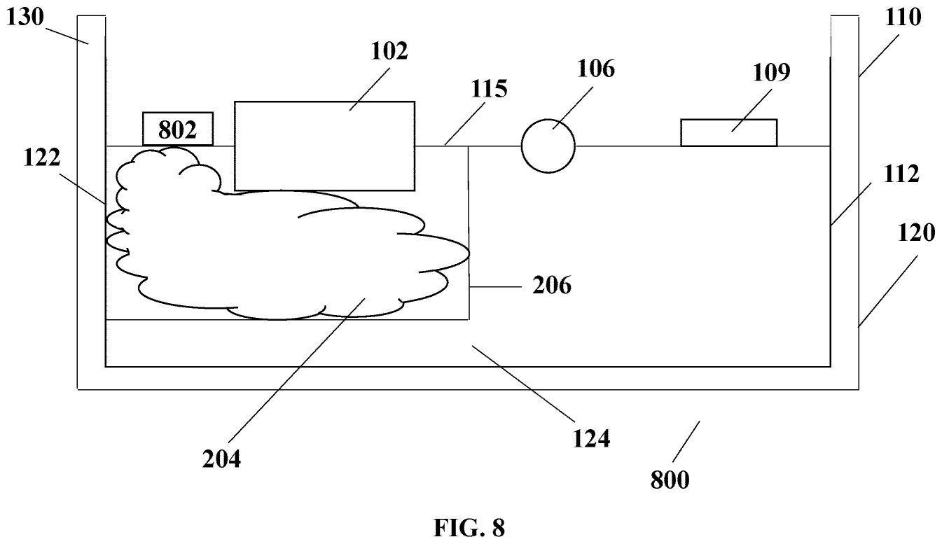

FIG. 8 is a side view (wall removed for visibility) of an environmental control and monitoring system 800 with a sensor system 802 in thermal contact with an environmental control element 204 through an intermediary according to some embodiments. In some embodiments, a sensor system 802 may be in thermal contact with an environmental control element 204 through an intermediary such as a retainer surface 124. In some embodiments, an environmental control element 204 may contact one side of a retainer surface 124 and a sensor system 602 may contact an opposing side of a retainer surface 124 such that a thermal mass comprises a portion of the retainer surface 124 in addition to the sensor system 602, environmental control element 204 and sample container 102. In some embodiments, the portion of the retainer surface 124 contacting the sensor system 802 and the environmental control element 204 may comprise a material having a high heat transfer efficiency.

In some embodiments, the sensor system 802 may span some or all of the retainer surface 124. In some embodiments, the sensor system 802 may be mounted to the retainer surface 124. In some embodiments, the sensor system 802 may be attached to the retainer surface 124 with adhesive, brackets, or any other known way to attach two objects.

In some embodiments, the environmental control and monitoring system 100 comprises an environmental control element 204, a sensor system 202, and a sample container 102 all being part of the same thermal mass. For example, in some embodiments, heat transfers easily between the environmental control element 204, the sensor system 202, and the sample container 102. In such embodiments, the environmental control element 204, the sensor system 202, and the sample container 102 are in thermal contact.

### Indicator Sensor Systems

In some aspects, indicator sensor systems are disclosed. Indicator sensor systems may be placed anywhere or on any surface of the environmental control and monitoring systems described herein. Indicator systems may be used alone or in conjunction with smart sensor systems. Any of the configurations depicted in FIGS. 1, 2, 6, 7, and 8 may comprise smart sensor systems, indicator sensor systems, or both.

In some embodiments, indicators may change appearance based on any a change in an environmental condition. For example, in some embodiments, an indicator may change appearance based on a temperature change, a humidity measurement, a pressure measurement, a contaminant, or any other measurable by a sensor. In some embodiments, an indicator may change appearance gradually as the duration of an exposure continues. In some embodiments, indicator systems may be activated by the patient upon packing of the semen or fertility sample either by button or pulling section.

In some embodiments, indicator systems may be incorporated into the thermal mass of the combined environmental control element and sample container. In some embodiments, the temperature of a sample container, environmental control element, and sensor system (e.g. an indicator sensor system) are about the same temperature due to efficient heat transfer due to being in thermal contact.

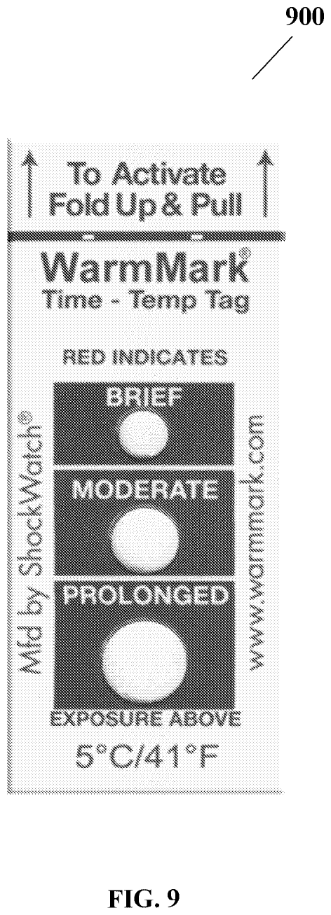

FIG. 9 is a temperature indicator 900 according to some embodiments. In some embodiments, indicators may include ingredients that chemically react based on temperature, humidity, pressure, or a contaminant. In some embodiments, indicators may be tuned to be reactive at specified temperatures. The example in FIG. 9 is reactive to exposure occurring below 4° C. In some embodiments, indicators can react to change appearance when a temperature goes above 37° C.

In some embodiments, an optimal temperature range for a sample may be between 4° C.-37° C., 5° C.-36° C., 6° C.-35° C., 7° C.-34° C., 8° C.-33° C., 9° C.-32° C., 10° C.-31° C., 11° C.-30° C., 12° C.-29° C., 13° C.-28° C., 14° C.-27° C., 15° C.-26° C., 16° C.-25° C., 17° C.-24° C., 18° C.-23° C., 19° C.-22° C., or 20° C.-21° C.

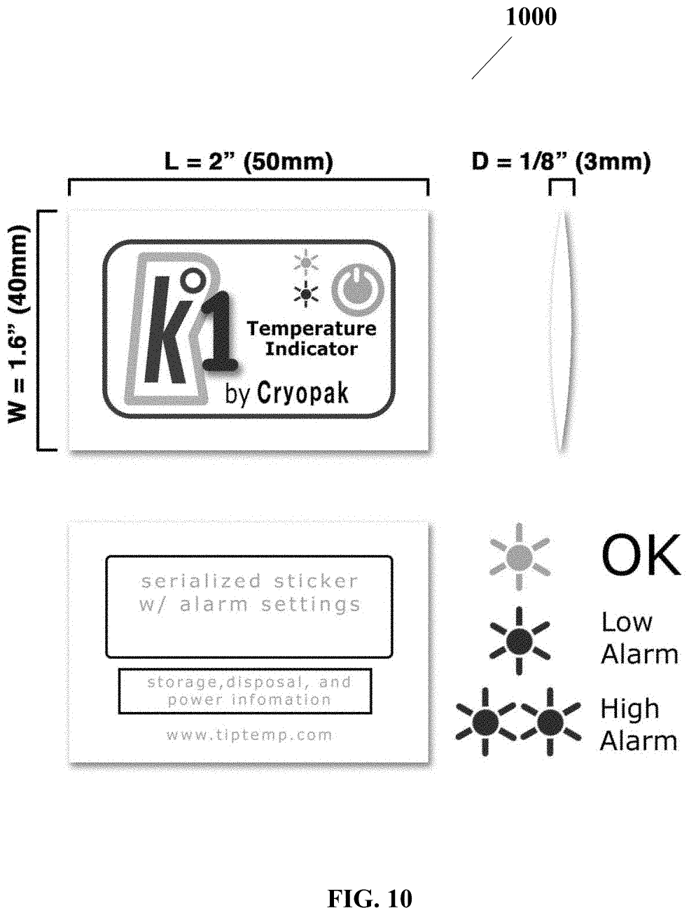

FIG. 10 is a temperature indicator 1000 according to some embodiments. In some embodiments, temperature indicators 1000 may comprise programmable electronic temperature indicators that do not necessitate use of chemically reactive powders. Commercially supplied temperature indicators can be turned for the specific application and work within a range. In some embodiments, the range may include 4° C.-37° C.

In some embodiments, an optimal temperature range for a sample may be between 4° C.-37° C., 5° C.-36° C., 6° C.-35° C., 7° C.-34° C., 8° C.-33° C., 9° C.-32° C., 10° C.-31° C., 11° C.-30° C., 12° C.-29° C., 13° C.-28° C., 14° C.-27° C., 15° C.-26° C., 16° C.-25° C., 17° C.-24° C., 18° C.-23° C., 19° C.-22° C., or 20° C.-21° C.

### Computer Systems

FIG. 5 is a block diagram that illustrates a computer system 500, upon which embodiments of the present teachings may be implemented. In various embodiments of the present teachings, computer system 500 can include a bus 502 or other communication mechanism for communicating information, and a processor 504 coupled with bus 502 for processing information. In various embodiments, computer system 500 can also include a memory, which can be a random-access memory (RAM) 506 or other dynamic storage device, coupled to bus 502 for determining instructions to be executed by processor 504. Memory also can be used for storing temporary variables or other intermediate information during execution of instructions to be executed by processor 504. In various embodiments, computer system 500 can further include a read only memory (ROM) 508 or other static storage device coupled to bus 502 for storing static information and instructions for processor 504. A storage device 510, such as a magnetic disk or optical disk, can be provided and coupled to bus 502 for storing information and instructions.

In some embodiments, computer system 500 can be coupled via bus 502 to a display 512, such as a cathode ray tube (CRT), liquid crystal display (LCD), or light emitting diode display (LED) for displaying information to a computer user. An input device 514, including alphanumeric and other keys, can be coupled to bus 502 for communicating information and command selections to processor 504. Another type of user input device is a cursor control 516, such as a mouse, a trackball or cursor direction keys for communicating direction information and command selections to processor 504 and for controlling cursor movement on display 512. This input device 514 typically has two degrees of freedom in two axes, a first axis (i.e., x) and a second axis (i.e., y), that allows the device to specify positions in a plane. However, it should be understood that input devices 514 allowing for 3-dimensional (x, y and z) cursor movement are also contemplated herein.

Consistent with certain implementations of the present teachings, results can be provided by computer system 500 in response to processor 504 executing one or more sequences of one or more instructions contained in memory 506. Such instructions can be read into memory 506 from another computer-readable medium or computer-readable storage medium, such as storage device 510. Execution of the sequences of instructions contained in memory 506 can cause processor 504 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 504 for execution. Such a medium can take many forms, including but not limited to, non-volatile media, volatile media, and transmission media. Examples of non-volatile media can include, but are not limited to, optical, solid state, magnetic disks, such as storage device 510. Examples of volatile media can include, but are not limited to, dynamic memory, such as memory 506. Examples of transmission media can include, but are not limited to, coaxial cables, copper wire, and fiber optics, including the wires that comprise bus 502.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 504 of computer system 500 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

### III. Methods

In some aspects of the disclosure methods for collecting, transporting, and analyzing fertility samples are described. In some embodiments, methods for environmental control and monitoring are described herein. In some embodiments, the methods describe herein determine whether a fertility sample may undergo fertility testing based on the environmental conditions to which the fertility sample was subjected.

### Methods Overview

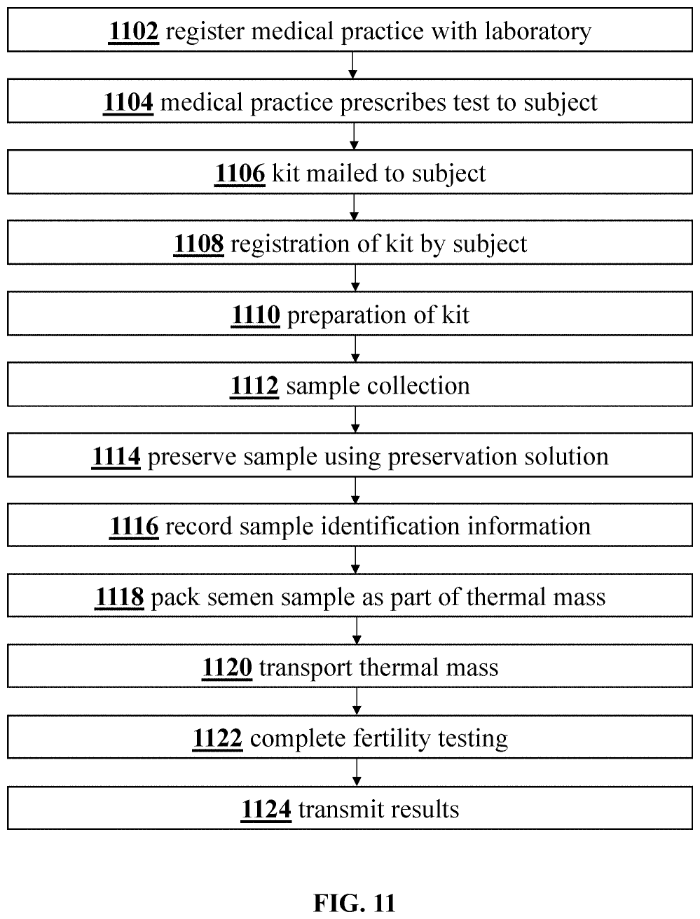

FIG. 11 is an illustration of a flow chart providing an overview of the technologies described herein according to some embodiments.

In some embodiments, the method may comprise a medical practice registering with a laboratory 1102. In some embodiments, registration allows for data flow between the sensor systems, remote servers, and clinic servers described herein. In some embodiments, each party may digitally identifiable. In some embodiments, registration may comprise onboarding activities including instructions on use of the kits described herein. In some embodiments, onboarding activities may include learning about the environmental conditions impacting a fertility sample's viability for fertility testing. In some embodiments, the strep of registration involves using a secure website.

In some embodiments, the method may comprise a medical practice prescribing a test to a subject 1104. In some embodiments, the test may include fertility testing. In some embodiments, fertility testing may include measuring a fluid volume, sperm motility testing, sperm concentration, or sperm morphology testing.

In some embodiments, the method may comprise mailing a kit 1106. In some embodiments, prior to mailing, a patient's information may be collected. In some embodiments, background information detailing the test may be presented to the subject. In some embodiments, shipping procedures may be presented to the subject. In some embodiments, package tracking information may be included with a kit. In some embodiments, an explanation of possible testing results and outcomes may be presented to the subject. In some embodiments, the steps described herein may be carried out over a web interface using a remote server and/or a clinic server.

In some embodiments, the method may comprise registering a kit 1108. In some embodiments, registration may include use of identification numbers or a QR code to identify a subject to a kit. In some embodiments, kit registration enables smart sensor system monitoring.

In some embodiments, the method may comprise preparing a kit 1110. In some embodiments, may comprise following instructs, wherein the instructions may be provided with the kit. In some embodiments, preparation may comprise activating an environmental control element. In some embodiments, preparation may comprise activating a sensor system. In some embodiments, activating a sensor system may comprise turning on a digital sensor system. In some embodiments, activating a sensor system may comprise pulling a tab or pushing a button to activate a sensor indicator.

In some embodiments, the method may comprise collecting a sample 1112. In some embodiments, collecting a sample may comprise a subject masturbating into a sample container under sterile conditions and attaching a lid after the sample has been deposited.

In some embodiments, the method may comprise preserving a sample using a preservation solution 1114 or fluid. In some embodiments, the method may comprise shipping the preservation fluid to a subject in a preservation container. In some embodiments, the preservation fluid may be added to the sample container after the sample has been deposited.

In some embodiments, the method may comprise recording a sample identification number 1116. In some embodiments, the identification number is configured to keep a subject anonymous while allowing for delivery of the correct result to the correct subject or patient.

In some embodiments, the method comprises packing the semen sample as part of a thermal mass 1118. In some embodiments, the thermal mass may comprise a sample container, an environmental control element, and a sensor system. In some embodiments, the purpose of the thermal mass is for accurate measurement of an environmental condition by the sensor system.

In some embodiments, the method comprises the step of transporting the thermal mass 1120. In some embodiments, the method comprises methods and systems disclosed herein to both control and monitor one or more environmental conditions during transport. In some embodiments, a log or record of the environmental condition(s) over time determines whether a fertility or semen sample is suitable for undergoing fertility testing.

In some embodiments, the method comprises completion of fertility testing 1122 after a determination has been made the sample is suitable for undergoing fertility testing. In some embodiments, the method comprises ordering and shipping another kit to the subject if a sample is determined to be unsuitable for undergoing fertility testing.

In some embodiments, the method comprises transmitting results 1124 to a clinic, subject, or another party or entity. In some embodiments, a web interface, registration process, and kit registration process enable matching of the correct subject to the correct result. In some embodiments, the method comprises sending results to the clinic and/or subject automatically from the remote server.

In some embodiments, the method comprises sets that may be carried out on a cell phone application in conjunction with a remote server. In some embodiments, the method comprises the step of the subject recording the time of shipment and the laboratory recording the time of receipt of the sample container. In some embodiments, a shipment duration determines a test sample's suitability for testing. In some embodiments, the method comprises the laboratory receiving the kit within 52 hours. In some embodiments, when the laboratory does not receive the kit within 52 hours another kit may be shipped to the subject for another attempt.

### Methods for Environmental Control and Monitoring

In some aspects of the disclosure, methods for environmental control and monitoring for transportation and analysis are disclosed. In some embodiments, the method comprises the step of transporting a thermal mass, wherein the thermal mass comprises a sensor system, an environmental control element, and a sample container. In some embodiments, the method comprises the step of controlling one or more environmental conditions of the thermal mass during transport using the environmental control element. In some embodiments, the method comprises the step of recording a plurality of measurements of the one or more environmental conditions during transport of the thermal mass to a log. In some embodiments, the method comprises the step of comparing the log to a reference to determine a condition within the sample container.

In some embodiments, the environmental control and monitoring method comprises the step of introducing a semen sample into the sample container prior to the transporting step. In some embodiments, introducing comprises a subject masturbating into the sample container in the comfort of their private residence.

In some embodiments, the environmental control and monitoring method may further comprise the step of registering the semen sample with a laboratory to associate a subject with the semen sample. In such embodiments, a laboratory will associate a set of environmental conditions occurring during transport with the subject's semen sample. In such embodiments, knowing the condition of the semen sample will result in either completion of fertility testing or contacting the subject for an additional semen sample.

In some embodiments, of the environmental control and monitoring method the one or more environmental conditions comprises temperature. Temperature may be a useful environmental condition to monitor because temperature changes over time correlate well with sperm health. Some sperm health indicators may include motility and morphology. When a laboratory knows the conditions under which a fertility same has traveled (e.g. a semen sample) it can make an informed decision as to whether the semen sample is in a condition for fertility testing. If a semen sample has undergone extreme temperature variations, a false indicator of fertility problems may occur during semen analysis.

In some embodiments, the method for environmental control and monitoring include the step of completing a plurality of measurements are recorded every 1, 2, 3, 4, 5, 6, 7, 8, 9, or 10 minutes. In such embodiments, temperatures may be recorded over time. When determining the health of a fertility sample knowing how far off-target a temperature was and for how long it persisted may determine whether the sample is in suitable condition for fertility testing.

In some embodiments of the environmental control and monitoring method the one or more environmental conditions comprises humidity. In some embodiments, humidity may indicate the readiness of a fertility sample to undergo fertility testing.

In some embodiments of the environmental control and monitoring method a log comprises a plurality of temperature and timestamp pairs. Stated different, in some embodiments, temperature measurements may be taken by a sensor and recorded at regular or irregular time intervals to create a history of temperatures. In some embodiments, knowing the history of one or more environmental conditions, such as a temperature condition, may determine a fertility sample's suitability to undergo fertility testing.

In some embodiments of the environmental control and monitoring method, the reference may comprise off-target temperature values. In some embodiments, the reference comprises exposure durations for the off-target temperature values. In some embodiments, the reference is based on research publications. In some embodiments, research publication data may be aggregated to produce a reference or set of references. Any known statistical model may be used in their production.

In some embodiments of the environmental control and monitoring method the reference is based on data acquired using the environmental control and monitoring method. In some embodiments, the methods described herein allow for collecting data, relating to a fertility sample, associated with changes in environmental conditions during transport such as temperature. In some embodiments, the fertility sample can be tested for sperm health. In some embodiments, testing parameters may include sperm motility, sperm count, sperm concentration, semen volume, or any other known metric to quantify or quantitate fertility health. In some embodiments, the data acquired may be compared to one or more of the metrics relating to sperm health to fine tune optimal transportation conditions.

In some embodiments, the methods and kits provided herein provide for measuring fertility conditions such as concentration of sperm in a sample, the progressive and non-progressive motility of the sperm in the sample, the total count of sperm in the sample, the total motility count, and the morphology of sperm as detailed in the guidelines published by the World Health Organization.

In some embodiments of the environmental control and monitoring method a condition determines whether the sample undergoes fertility testing and is based on sperm count, motility count, or morphology.

In some embodiments of the method of environmental control and monitoring, the step of controlling comprises causing a phase transition material of the environmental control element to undergo a phase transition. In some embodiments, the environmental control element comprises a gel pack.

In some embodiments of the method of environmental control and monitoring, a log comprises a temperature indicator. In some embodiments, the temperature indicator comprises an adhesive strip including a temperature sensitive region, wherein the temperature sensitive region gradually changes appearance based on duration of exposure to off-target temperature values.

In some embodiments of the environmental control and monitoring method, the method further comprises the steps of storing the log on a data store in digital format, transmitting the log from the data store to a remote server, and completing the comparing step on the remote server.

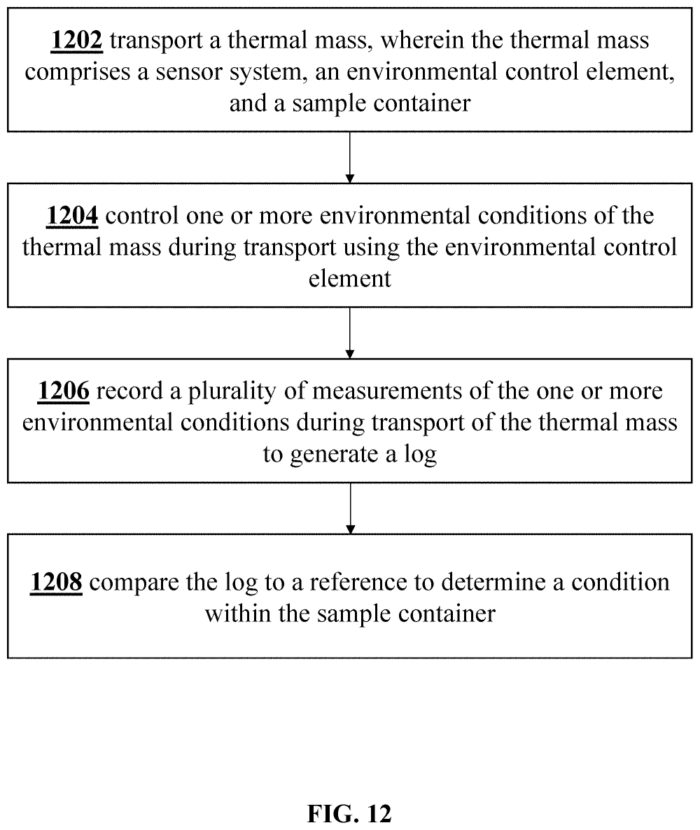

FIG. 12 is a flow chart illustrating a method of controlling and monitoring environmental conditions of a thermal mass (e.g. contents within a package) according to some embodiments. In some embodiments, the method may comprise the step of transporting a thermal mass, wherein the thermal mass comprises a sensor system, an environmental control element, and a sample container 1202. In some embodiments, the method may comprise the step of controlling one or more environmental conditions of the thermal mass during transport using the environmental control element 1204. In some embodiments, the method may comprise the step of recording a plurality of measurements of the one or more environmental conditions during transport of the thermal mass to generate a log 1206. In some embodiments, the method may comprise the step of comparing the log to a reference to determine a condition within the sample container 1208.

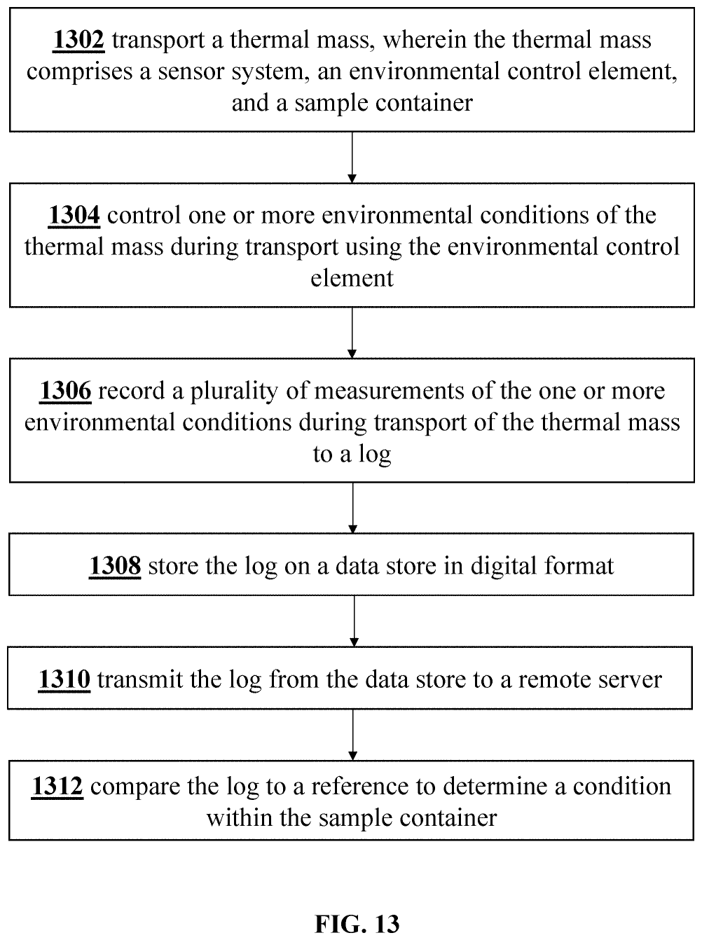

FIG. 13 is a flow chart illustrating a method of controlling and monitoring environmental conditions, using a smart sensor system, of a thermal mass (e.g. contents within a package) according to some embodiments. In some embodiments, the method may comprise the step of transporting a thermal mass, wherein the thermal mass comprises a sensor system, an environmental control element, and a sample container 1302. In some embodiments, the method may comprise the step of controlling one or more environmental conditions of the thermal mass during transport using the environmental control element 1304. In some embodiments, the method may comprise the step of recording a plurality of measurements of the one or more environmental conditions during transport of the thermal mass to a log 1306. In some embodiments, the method may comprise the step of storing the log on a data store in digital format 1308. In some embodiments, the method may comprise the step of transmitting the log from the data store to a remote server 1310. In some embodiments, the method may comprise the step of comparing the log to a reference to determine a condition within the sample container 1312.

In some embodiments, the transporting step may include travel over 10 miles, 20 miles, 30 miles, 40 miles, 50 miles, 60 miles, 70 miles, 80 miles, 90 miles, or 100 miles. In some embodiments, the transporting step may include travel over 10 km, 20 km, 30 km, 40 km, 50 km, 60 km, 70 km, 80 km, 90 km, or 100 km.

In some embodiments the transporting step may include traveling 52 hours or less. In some embodiments the transporting step may include traveling between about 1-2, 2-3, 3-4, 5-6, 6-7, 7-8, 8-9, 9-10, 10-11, 11-12, 12-13, 13-14, 14-15, 15-16, 16-17, 17-18, 18-19, 20-21, 21-22, 22-23, 23-24, 24-25, 25-26, 26-27, 27-28, 28-29, 29-30, 30-31, 31-32, 32-33, 33-34, 34-35, 35-36, 36-37, 37-38, 38-39, 39-40, 41-42, 42-43, 43-44, 44-45, 45-46, 46-47, 47-48, 48-49, 49-50, 50-51, or 51-52 hours, or any combination thereof.

### IV. Kits

In some aspects of the disclosure, kits are described. In some embodiments, the kits may be used in carrying out some of the methods described herein.

In some embodiments, a kit may comprise a sample container for storing a semen sample, an environmental control element for controlling a temperature of the sample container, and a sensor system for monitoring the temperature of the sample container.

In some embodiments, sensor systems in a kit may further comprise a temperature sensor, a data store for recording measurements produced by the temperature sensor, and a data port for transmitting the measurements.

In some embodiments, a kit may further comprise a positioning retainer for restraining movement of the sample container, the environmental control element, the sensor system relative to one another.

In some embodiments, a kit may further comprise a preservation container comprising a preservation fluid. In some embodiments, the preservation fluid or sperm preservation fluid may comprise pH stabilizing agents and ions and nutrients to promote sperm health. In some embodiments, the preservation fluid may comprise salts, sugar, and/or an antibiotic agent.

In some embodiments, a sensor system may comprise an appearance changing temperature indicator. In some embodiments, the appearance change involves a material going through a chemical transition after exposure to an off-target temperature or pH.

In some embodiments, the kit may comprise a sterilized pouch for placement and containment of the sample container during transport.

In some embodiments, the environmental control element comprises a phase transition material. In some embodiments, the phase transition material is contained within a plastic bag and may be referred to as a gel pack. In some embodiments, the environmental control element has been optimized to operate within a validated range. In some embodiments, the validated range may be derived from literature detailed optimal environmental conditions for healthy sperm.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

In describing the various embodiments, the specification can have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences can be varied and still remain within the spirit and scope of the various embodiments.

1. An environmental control and monitoring system for transportation and analysis of a semen sample, the system comprising:
a sample container, configured to contain a semen sample;
an environmental control element configured to operate within an optimal range for semen viability; and
a sensor system for monitoring an environmental condition of the sample container, wherein the sensor system is in thermal contact with at least one of the sample container or the environmental control element.

2. The environmental control and monitoring system of claim 1, wherein the system further comprises:
a positioning retainer for restraining movement of the sample container, the environmental control element, and the sensor relative to one another.

3. The environmental control and monitoring system of claim 2, wherein the positioning retainer comprises a packing material.

4. The environmental control and monitoring system of claim 1, wherein the sensor system further comprises:
a sensor for measuring the environmental condition;
a data store in electronic communication with the sensor, wherein the data store is configured to store measurements from the sensor; and
a data port in electronic communication with the data store.

5. The environmental control and monitoring system of claim 4, wherein the sensor comprises a temperature sensor and the environmental condition comprises temperature.

6. The environmental control and monitoring system of claim 4, wherein the sensor comprises a humidity sensor and the environmental condition comprises humidity.

7. The environmental control and monitoring system of claim 4, wherein the sensor measures the environmental condition at least one time every minute.

8. The environmental control and monitoring system of claim 4, wherein measurements from the sensor system are stored on the data store.

9. The environmental control and monitoring system of claim 4, wherein the data port electronically communicates measurements from the sensor system to a remote server.

10. The environmental control and monitoring system of claim 9, wherein the measurements are stored on the data store prior to being transmitted to the remote server.

11. The environmental control and monitoring system of claim 10, wherein the remote server comprises:
a server data port for sending and receiving data, wherein the data comprises the measurements;
a server data store in electronic communication with the server data port for storing the data; and
a sever processor for analyzing the data.

12. The environmental control and monitoring system of claim 1, wherein the environmental control element comprises a phase transition material.

13. The environmental control and monitoring system of claim 12, wherein the phase transition material is configured to go through a phase change within the optimal temperature range.

14. The environmental control and monitoring system of claim 1, wherein the optimal range comprises temperatures ranging from 4° C. to 37° C.

15. The environmental control and monitoring system of claim 2, further comprising a container wall surrounding an internal cavity, wherein the positioning retainer fits within the internal cavity, wherein the cavity is surrounded by the container wall.

16. The environmental control and monitoring system of claim 1, wherein the sample container holds a semen preservation solution.

17. The environmental control and monitoring system of claim 11, wherein the data port and the server data port each comprise a wireless adaptor.

18. The environmental control and monitoring system of claim 1, wherein the sensor system comprises a temperature indicator.

19. The environmental control and monitoring system of claim 18, wherein the indicator displays a level of exposure.

20. The environmental control and monitoring system of claim 18, wherein the temperature indicator is in physical and thermal contact with an exterior surface.

21. The environmental control and monitoring system of claim 18, wherein the temperature indicator is in physical and thermal contact with an interior surface.

22. The environmental control and monitoring system of claim 18, wherein the temperature indicator is in physical and thermal contact with a retainer surface.

23-61. (canceled)
